(19) United States
(12) Patent Application Publication
(10) Pub. No.: US 2025/0288870 A1
(43) Pub. Date: Sep. 18, 2025

(54) DEVICE AND METHODS FOR MEASURING SHOULDER STRENGTH

(71) Applicant: Titin KM Biomedical Corp., Bozeman, MT (US)

(72) Inventors: Kole Mickolio, Billings, MT (US); Rory Maughan, Bozeman, MT (US); Jordan Brett Restifo, Parkland, FL (US)

(73) Assignee: Titin KM Biomedical Corp., Bozeman, MT (US)

(21) Appl. No.: 19/227,148

(22) Filed: Jun. 3, 2025

Related U.S. Application Data
(63) Continuation of application No. PCT/US2023/082151, filed on Dec. 1, 2023.
(60) Provisional application No. 63/430,757, filed on Dec. 7, 2022.
(60) Provisional application No. 63/526,385, filed on Jul. 12, 2023.

(51) Int. Cl. A63B 24/00; A63B 21/16; A63B 23/12

(52) U.S. Cl. CPC A63B 24/0062; A63B 21/1609; A63B 23/1272; A63B 2220/17

(57) ABSTRACT

Exercise devices can provide multidirectional and dynamic resistance to movement of a user, such that complex movements can be evaluated and assessed. An exercise apparatus includes a shaft, a user engagement structure coupled to the shaft, a position sensor coupled to the shaft configured to detect a position of the shaft, and a processor operatively coupled to the position sensor. The processor is configured to receive position data from the position sensor, calculate position coordinates based on the position data that are representative of a position of the user engagement structure, determine whether the position coordinates are within a previously-defined region, and count a repetition when the position coordinates are within the previously-defined region.

### CROSS-REFERENCED TO RELATED APPLICATIONS

This application is a continuation of International Patent Application No. PCT/US2023/082151, filed Dec. 1, 2023, which claims the benefit of U.S. Provisional Application Nos. 63/526,385, filed Jul. 12, 2023, and 63/430,757, filed Dec. 7, 2022, each of which is incorporated by reference herein.

### FIELD

The present disclosure relates generally to exercise equipment, and more particularly to exercise equipment for measuring shoulder strength.

### BACKGROUND

Physical therapy treatment and the exercises used for shoulder strengthening are currently hampered by a lack of dynamic, weight-bearing equipment, that can isolate the shoulder joint in 360 degrees of motion. Because surgical procedures alone are unable to fully repair one's shoulder, physicians and patients are left reliant on conventional exercise equipment for rehabilitation. The existing shortcomings in shoulder rehabilitation, especially post-surgery rehabilitation, are attributable to the limited utility of elastic bands, medicine balls, dumbbells, and other conventional weight-room equipment typically used to strengthen the shoulder. Conventional exercise equipment, for instance, only allow for resistance in one plane of shoulder-joint motion at any one time, such as motion in the coronal plane about an anterior-posterior axis, and motion in the sagittal plane about a medial-lateral axis. A shoulder strengthening system that can address the significant lack of dynamic weight bearing equipment in the current field of physical therapy and shoulder recovery is needed.

### SUMMARY

According to an aspect of the disclosed technology, an exercise apparatus includes a shaft, a user engagement structure coupled to the shaft, a position sensor coupled to the shaft configured to detect a position of the shaft, and a processor operatively coupled to the position sensor. The processor is configured to receive position data from the position sensor, calculate position coordinates based on the position data that are representative of a position of the user engagement structure, determine whether the position coordinates are within a previously-defined region, and count a repetition when the position coordinates are within the previously-defined region.

The foregoing and other objects, features, and advantages of the technology will become more apparent from the following detailed description, which proceeds with reference to the accompanying figures.

### BRIEF DESCRIPTION OF THE DRAWINGS

FIG. 1A is a perspective view of an exercise device, according to one example.

FIG. 1B is a perspective view of an exercise device, according to one example.

FIG. 2 is a perspective view of the exercise device of FIG. 1A, with a cover removed from a base of the exercise device.

FIG. 3 is a perspective view of the exercise device of FIG. 1A showing additional details of the base.

FIG. 4 is another perspective view of the exercise device of FIG. 1A showing additional details of the base.

FIG. 5 is a side view of a shaft of the exercise device of FIG. 1A.

FIG. 6 is a schematic view of an example of a path in three-dimensional space of a repetition of an exercise performed using an exercise device.

FIG. 7 is a flow chart outlining an exemplary method of counting a repetition of an exercise performed using an exercise device.

FIG. 8 is a flow chart outlining an exemplary method of counting a repetition of an exercise performed using an exercise device.

FIG. 9 is a flow chart outlining an exemplary method of counting a repetition of an exercise performed using an exercise device.

FIG. 10 is a flow chart outlining an exemplary method of adjusting a resistance of an exercise device.

FIG. 11 is a flow chart outlining an exemplary method of evaluating a repetition of an exercise performed using an exercise device.

FIG. 12 depicts an exemplary computing system for implementing the disclosed technology.

FIG. 13 illustrates an example output on a graphical user interface of an exercise device.

FIG. 14 illustrates an example output on a graphical user interface of an exercise device.

FIG. 15 is a flow chart outlining an exemplary method of evaluating a repetition of an exercise performed using an exercise device.

### DETAILED DESCRIPTION

### General Considerations

The systems, apparatus, and methods described herein should not be construed as limiting in any way. Instead, the present disclosure is directed toward all novel and non-obvious features and aspects of the various disclosed examples, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present, or problems be solved. Any theories of operation are to facilitate explanation, but the disclosed systems, methods, and apparatus are not limited to such theories of operation.

In some examples, values, procedures, or apparatus are referred to as “lowest,” “best,” “minimum,” or the like. It will be appreciated that such descriptions are intended to indicate that a selection among many used functional alternatives can be made, and such selections need not be better, smaller, or otherwise preferable to other selections.

As used in the application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,” “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Examples of the Disclosed Technology

There is a growing consensus among physical therapists and medical practitioners that the use of elastic bands and other conventional equipment used for shoulder rehabilitation show a lack of efficacy. The shoulder joint is a ball-in-socket joint that has nearly 360 degrees of motion in multiple planes, making it the most dynamic and unstable joint in the body. Indeed, the most common muscles and joint injuries among athletes and the general population are the various muscles that attach around the shoulder joint as well as the surrounding cartilage and the labrum. For this reason, an exercise system which can advance the current state of available equipment for shoulder rehabilitation is needed.

### Example Exercise Device

Systems of the present invention can include exercise devices that have multiple degrees of freedom and can provide resistance at each physiological plane and angle. The exercise devices enable complex movements (such as movements that mimic e.g., throwing a ball, swinging a golf club, kicking a ball, other complex functional movements, etc.) to be performed and assessed. Exercise devices can include a shaft coupled to at least one joint that enables multidirectional movement of the shaft and a resistance system that provides multidirectional and dynamic resistance to the shaft. A user can engage with the shaft, or a user engagement structure coupled to the shaft, to complete an exercise, for example, by moving the shaft along a path. Examples of exercise devices are further described in International Patent Application No. PCT/US2022/023150 and International Patent Application No. PCT/US2022/045755, the entire disclosures and teachings of which are incorporated herein by reference.

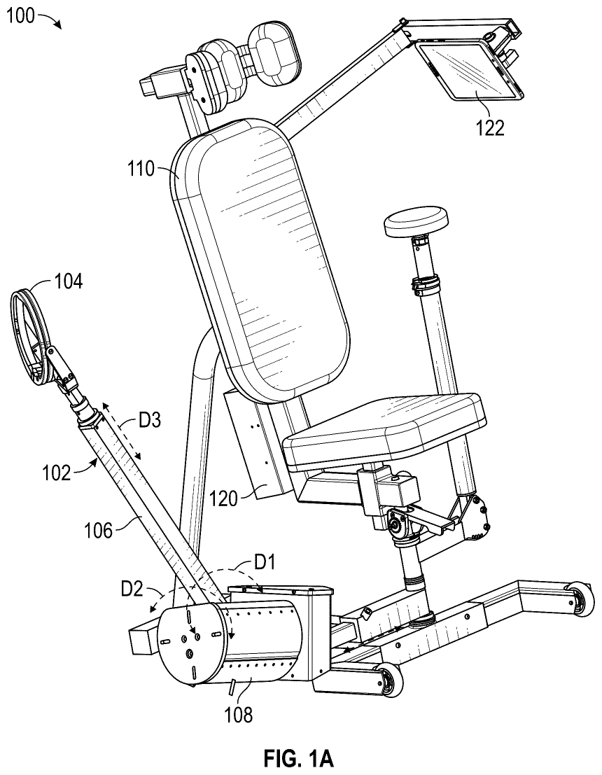

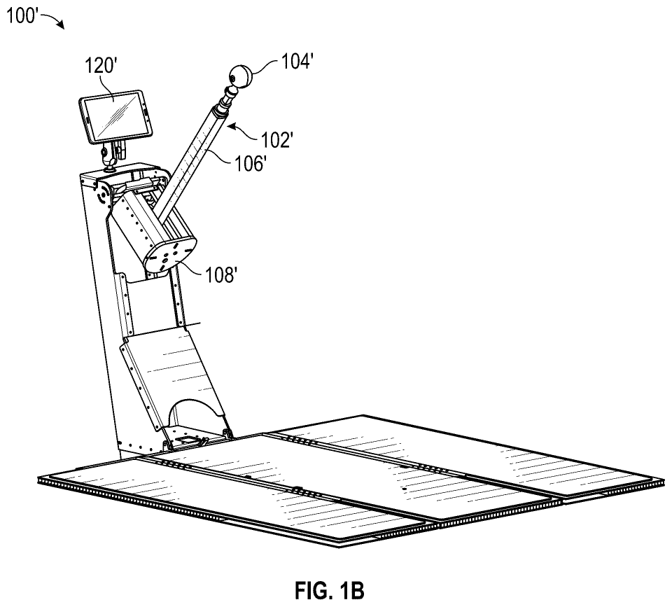

FIG. 1A illustrates an example of an exercise device 100. The exercise device 100 includes a shaft assembly 102 having a user engagement structure 104 coupled to a distal end of a telescoping shaft 106. In some examples, the user engagement structure 104 can include a handle for the user to selectively grip or grasp, a brace (e.g., a wrist brace) for the user to be securely attached to, or other structures for the user to engage with. In the illustrated example, the user engagement structure 104 includes a ring structure to which a brace (e.g., a wrist brace) can be rotatably mounted, such that the wrist of the user can rotate and pivot about multiple axes within the ring structure. In some examples, the user engagement structure 104 can replicate athletic equipment (e.g., a ball, a handle of a racket or club, etc.), occupational tools (e.g., a hammer, a drill, etc.), or the like. The proximal end of the telescoping shaft 106 is coupled to a base 108. In some examples, the base 108 of the shaft assembly 102 can be coupled to a chair 110 that allows the user to sit while performing exercises using the exercise device 100.

In some examples, an exercise device does not include a chair. FIG. 1B shows an example of an exercise device 100′ that is similar to exercise device 100 but does not include a chair. For example, similar to exercise device 100, exercise device 100′ includes a shaft assembly 102′ having a user engagement structure 104′ (e.g., a ball) coupled to a distal end of a telescoping shaft 106′. In some examples, a base 108′ of the shaft assembly 102′ can be coupled to a platform 109 such that a user can be standing when performing exercises using the exercise device 100′. In some examples, the base 108′ can be rotated relative to the platform 109 about a joint to better accommodate various complex movements while the user engages with the user engagement structure 104′. In some examples, as shown, the base 108′ (e.g., the proximal end of the shaft assembly 102′) can be vertically spaced apart from the ground in some examples.

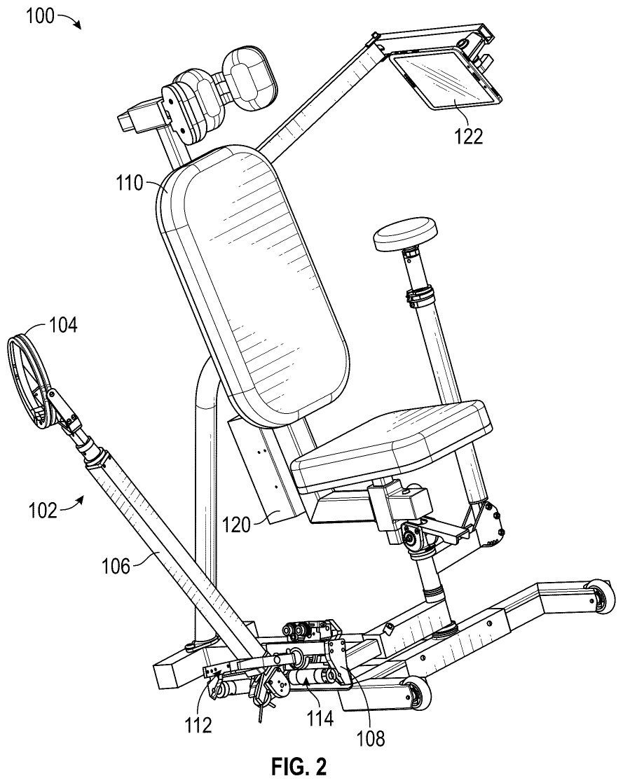

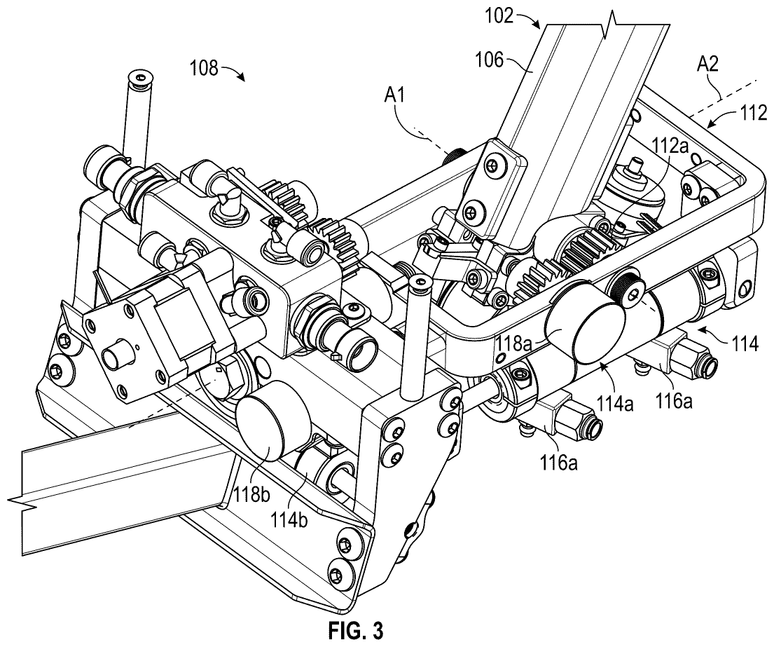

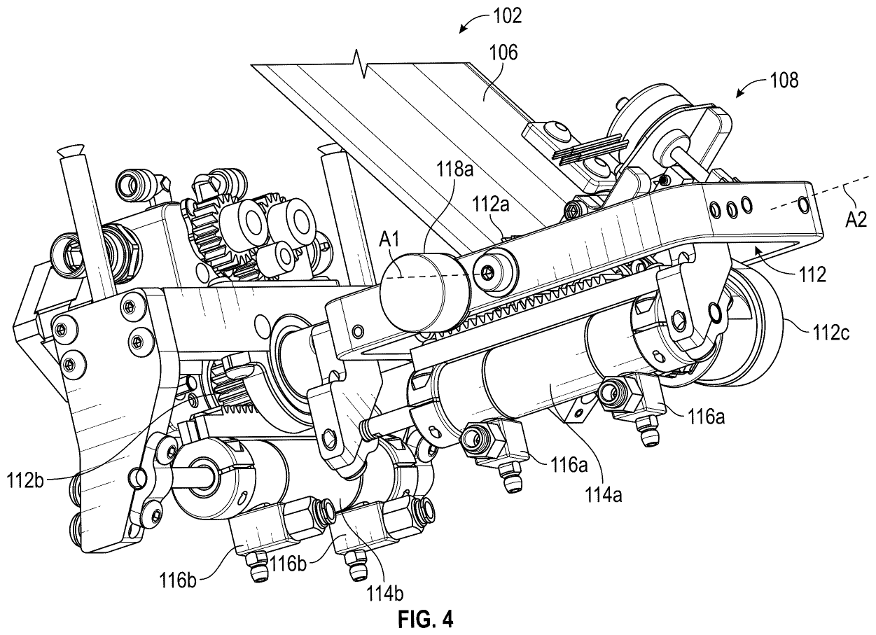

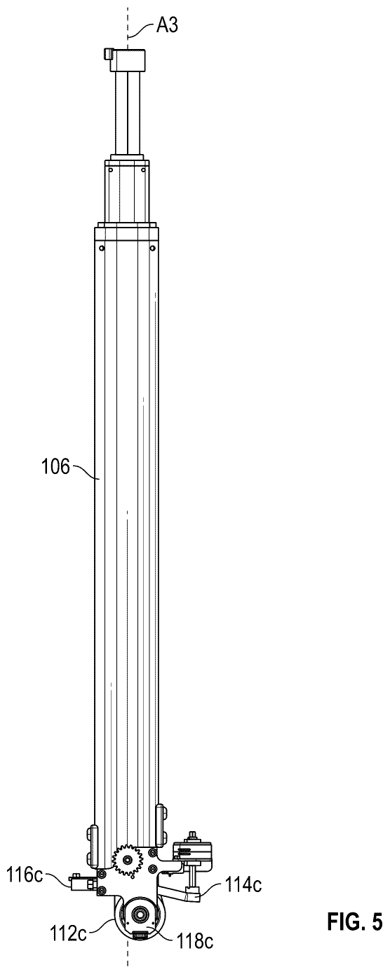

FIGS. 2-5 show additional details of the exercise device 100, although it should be appreciated that these features may be included in exercise device 100′. The base 108 can house a joint assembly 112 that enables multidirectional movement of the telescoping shaft. As shown in FIGS. 3-4, the joint assembly 112 can include a first joint 112a that enables rotation of the shaft assembly 102 about a first axis A1 (e.g., in the direction D1 in FIG. 1) and a second joint 112b that enables rotation of the shaft assembly 102 about a second axis A2 that is orthogonal to the first axis A1 (e.g., in the direction D2 in FIG. 1). As shown in FIG. 5, the telescoping shaft 106 can include a telescopic joint 112c (e.g., a pulley system, etc.) that enables a length of the telescoping shaft 106 to be adjusted along a longitudinal axis A3 of the shaft 106 (e.g., in the direction D3 in FIG. 1).

A resistance mechanism 114 can be coupled to the shaft assembly 102. The resistance mechanism 114 can include multiple brakes 114a, 114b, 114c. Each brake 114a, 114b, 114c is coupled to one of the joints 112a, 112b, 112c, respectively. The brakes 114a, 114b, 114c can be configured to apply a resistance or braking force to the joints 112a, 112b, 112c, thereby opposing and/or resisting the multidirectional movement of the shaft assembly 102. In the illustrated example, the brakes 114a, 114b comprise dual-action hydraulic cylinders and brake 114c comprises a motorized arm brake, although other configurations are contemplated. Each brake 114a, 114b, 114c can include (and/or be coupled to) one or more resistance sensors configured to detect an amount of resistance or braking force applied to each joint 112a, 112b, 112c, respectively. For example, pressure sensors 116a, 116b are coupled to the hydraulic brakes 114a, 114b, respectively, to detect the pressure of the hydraulic fluid used to resist motion of the joints 112a, 112b. As shown, two pressure sensors are coupled to each dual action hydraulic cylinder to detect hydraulic pressure in both directions. A load cell 116c can be coupled to the motorized arm brake 114c to detect an amount of force applied to the joint 112c.

The exercise device 100 also includes at least one position sensor that is configured to detect and/or measure the position of the shaft assembly 102 relative to the base 108. For example, the exercise device 100 can include multiple position sensors 118a, 118b, 118c coupled to each joint 112a, 112b, 112c, respectively. In some examples, as depicted in FIGS. 3-4, the position sensors 118a and 118b can be rotational position sensors (e.g., a digital and/or analog rotary encoder) and can be configured to measure a rotational position of the shaft assembly 102 relative to the first axis A1 and the second axis A2. For example, rotation of the first and second position sensors 118a and 118b corresponds to rotation of the shaft assembly 102 about a respective axis A1, A2, such that the position sensors 118a, 118b can measure a corresponding rotational movement of the shaft assembly 102. As shown in FIG. 5, the position sensor 118c can be coupled to the telescoping shaft 106. For example, the position sensor 118c (e.g., a rotary sensor, an optical encoder, etc.) can be coupled to the joint 112c, and can be configured to track and measure the telescoping position/motion of the telescoping shaft 106 relative to the base 108 (e.g., to determine a position to which the shaft 106 has been extended or retracted). The position sensor 118c can, for instance, detect a rotational position of a roller or pulley of the joint 112c to measure a length of belt or cable that has been unwound from the roller or pulley. In this way, the position sensor 118c can be used to determine a length to which the telescoping shaft 106 of the shaft assembly 102 has been extended (e.g., a distance between the user engagement structure 104 and the base 108).

Other types and/or configurations of sensors can be used to track the position of the shaft assembly 102. For example, one or more of the position sensors 118a, 118b, 118c can be linear position sensors that detect a position of the shaft assembly 102 based on a linear position of a component and/or portion of the exercise device 100 (e.g., joints 112a, 112b, 112c, a portion of the shaft assembly 102, etc.). In some instances, for example, the position sensors 118a, 118b can be linear position sensors and can be configured to measure a position of the shaft assembly 102 based on a linear position of each joint 112a, 112b relative to its respective supporting structure. The linear position can be used to determine the rotational positioning of the shaft assembly 102 about the first axis A1 and the second axis A2.

The exercise device 100 can include a computing system 120 operatively coupled to each of the sensors (e.g., sensors 116a-c, sensors 118a-c, etc.) and the resistance mechanism 114 via a connection (e.g., a wired or wireless connection, such as Bluetooth, etc.). In some examples, the computing system 120 can include a display 122 (e.g., a tablet, an LCD display, etc.) to output data from the sensors, the resistance mechanism, and/or other data related to the exercise device 100, as described in more detail below.

### Position Tracking

Each of the position sensors 118a, 118b, 118c is operatively coupled to the computing system 120. The output from the position sensors 118a, 118b, 118c can be transmitted to the computing system 120 and converted into position data. For example, the output from the sensors 118a, 118b, 118c can be received by the computing system 120 and used to calculate and/or determine a position of the distal end of the shaft assembly 102, and in particular, the user engagement structure 104 of the exercise device 100. In other words, the relative position data from each of the sensors 118a, 118b, 118c can be combined to create the absolute position of the user engagement structure 104 at any given moment.

Position data can represent the location of the user engagement structure 104 in three-dimensional space. For example, the position data can comprise coordinates, such as cartesian coordinates (x, y, z), spherical coordinates (r, θ, φ), or other coordinate systems, such that the position of the user engagement structure 104 can be tracked. The three-dimensional space is defined to encompass all possible locations of the user engagement structure 104 as the user moves the shaft assembly 102 (e.g., during an exercise). In some examples, the origin of the coordinate system within the three-dimensional space is located at a fixed position on the exercise device 100 (e.g., the base 108, the chair 110, etc.). In some examples, the origin can be located such that certain axes of the coordinate system (e.g., x-axis, y-axis, etc.) are aligned with the first axis A1 and/or the second axis A2 of the exercise device. In other examples, the origin of the coordinate system can be defined based on an initial starting position of the user engagement structure 104 and/or the shaft assembly 102.

The computing system 120 can be configured to calculate position coordinates of the user engagement structure 104 as the user moves the shaft assembly 102, for example, during an exercise. As one example, the computing system 120 can calculate the length of the telescoping shaft 106 (e.g., a distance between the base 108 and the user engagement structure 104) based on data from the position sensor 118c and determine the rotational positioning of the telescoping shaft 106 using Euler angles, based on data from the position sensors 118a, 118b. Using the length and the angles of the telescoping shaft 106 (e.g., relative to the base 108, etc.) associated with time data, the computing system 120 can calculate position coordinates of the user engagement structure 104 at a particular time during the exercise. In some examples, each position can be linked with time data (e.g., a timestamp, etc.) indicative of the time at which the position data was sensed by the sensors 118a, 118b, 118c. The position coordinates can be calculated in real time (or near real time) such that position data and/or feedback can be presented to the user (e.g., via the display 122) as the user performs an exercise. In some examples, the position of the base 108 (e.g., relative to the chair 110) can be used to globally skew, transpose, and/or rotate the position coordinates so that the position of the user engagement structure 104 can be correctly represented relative to the user.

In some examples, the position data can also be used to determine various vectors associated with the movement of the user engagement structure 104. For example, based on the position coordinates and time data, the computing system 120 can determine a direction vector, a velocity vector, an acceleration vector, and/or other vectors at each position as the shaft assembly 102 is moved along a path. These vectors can be useful when counting repetitions as well as determining other performance metrics, as discussed in more detail below.

In some examples, the computing system 120 can store the position data to enable a user (e.g., a patient, a physical therapist, etc.) to review the performance of exercises in a particular session, etc. In some examples, the position data (and other metrics described herein) can be stored in a secure profile that is specific to the user, such that the user can access and track performance over time in the user profile (e.g., via the display 122).

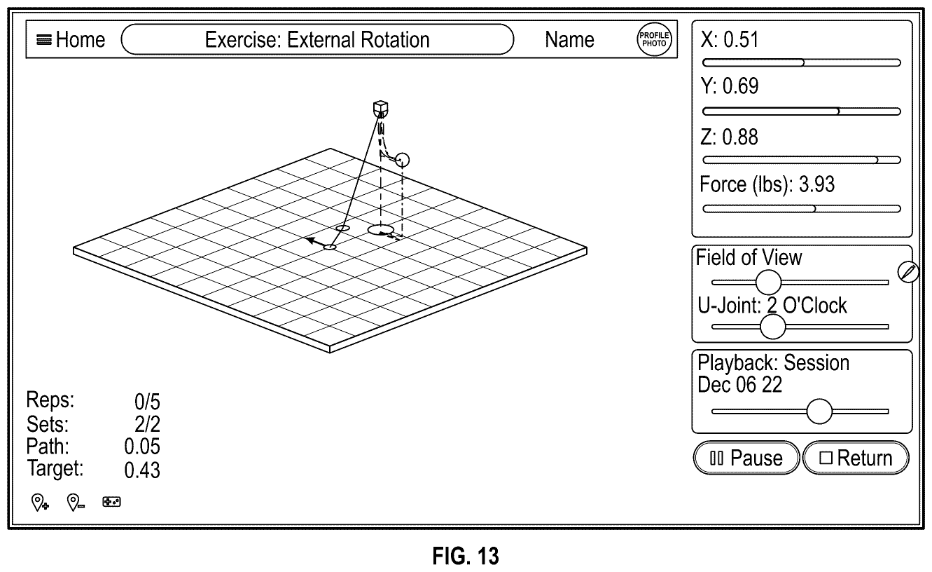

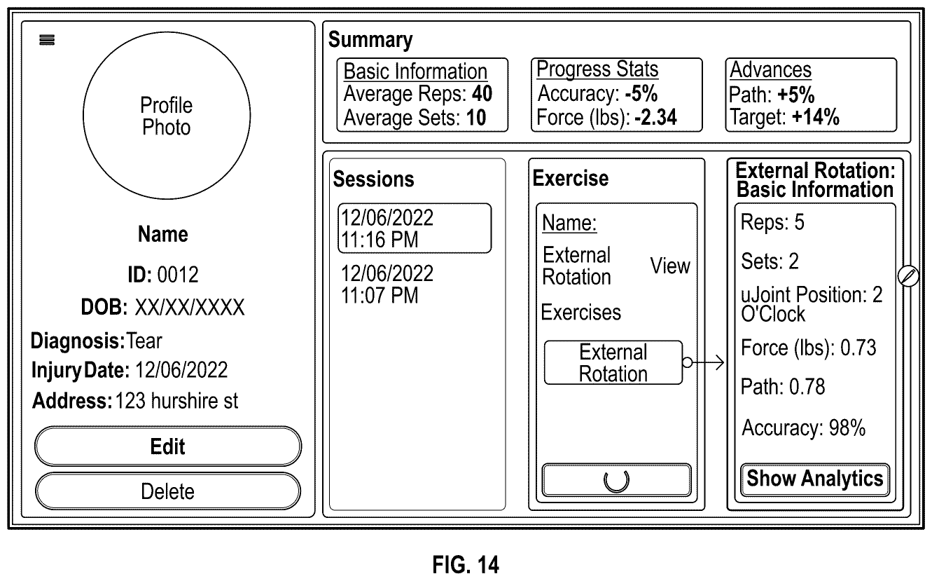

Example output data is shown in FIGS. 13-14 that can be presented via the display 122. FIG. 13 illustrates output corresponding to a user performing an exercise (e.g., external rotation) using the exercise device 100, including a three-dimensional path of the user engagement structure 104, current position coordinates of the user engagement structure 104, force output, and other data. An example of a user profile is shown in FIG. 14.

### Counting Repetitions

To perform a given exercise using exercise device 100, a user moves the user engagement structure 104 of the shaft assembly 102 along a path. The user may repeatedly move the user engagement structure 104 along the same path (or approximately along the same path) to complete a certain number of repetitions of the exercise, for example, as part of a strengthening and/or rehabilitation exercise regimen. In some examples, the user may move the user engagement structure 104 along randomized paths, such that each repetition follows a different path than one or more of the prior repetitions.

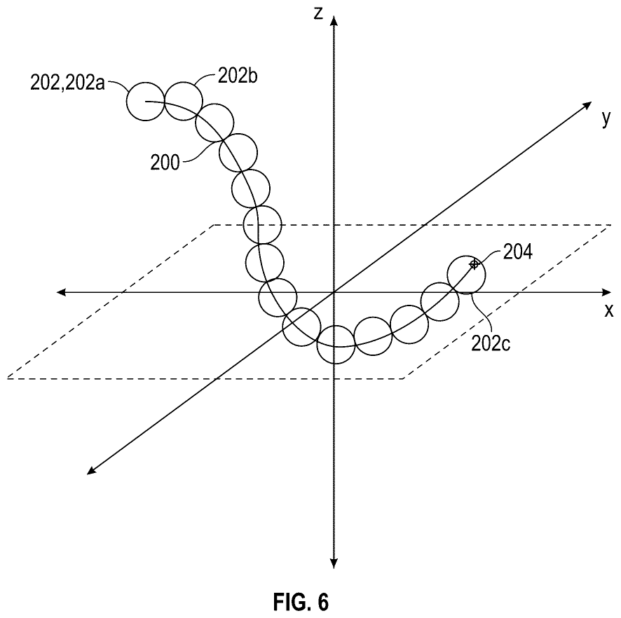

It may be useful to track the number of repetitions that the user performs of the given exercise. To determine whether a user has completed a repetition of a given exercise, position data from the sensors 118a, 118b, 118c can be used. In some examples, the position data can be used to create a three-dimensional plot of the path 200 of the user engagement structure 104, as shown in FIG. 6. A series of regions 202 can be defined around the data points (or coordinates) of the path 200. Each region 202 represents a three-dimensional space around one or more data points on the path 200, with each data point representative of the position of the user engagement structure 104 in space at a particular time.

A first region 202a can be defined around the start point or initial position of the path 200. As the user engagement structure 104 is moved, data points can be plotted to define the path 200. When the user engagement structure 104 is moved to a position that is outside of the first region 202a, a second region 202b can be defined that is adjacent to the first region 202a. A series of regions 202 can be created (e.g., in real time) as the user engagement structure 104 moves through space.

In some examples, the path 200 can include an indicator 204 that is representative of a current (or most recent) position of the user engagement structure 104. In particular, the indicator 204 can be located at the position coordinates that have the most recent time value. In some examples, the region 202 in which the user engagement structure 104 is currently positioned (e.g., region 202c in FIG. 6), as represented by indicator 204, is considered an “active” or “current” region. When the indicator 204 exits the active region (e.g., when the user engagement structure 104 is moved a certain distance away), the computing system 120 determines whether the indicator 204 is entering a space that has not yet been defined as a region or whether the indicator 204 is entering an existing or previously defined region (e.g., the regions 202a-202b in FIG. 6). When the indicator 204 is entering a space that has not yet been defined as a region (e.g., the user engagement structure 104 is moving to a new physical location), the computing system 120 creates a new region for the path 200 and the new region is considered the active region.

When the indicator 204 is moved from an active region (e.g., the region 202c in FIG. 6) into an existing region (e.g., the region 202a, region 202b, etc.) rather than into a new or previously undefined region, the computing system 120 counts a repetition, as the exercise is considered to be complete. Considering a repetition complete when the indicator 204 returns to any one of the existing regions (e.g., the regions 202a-202b) enables a repetition to be counted for simple and complex movements, including movements and/or exercises that return along the same path (e.g., a bicep curl) as well as for movements and/or exercises that do not necessarily return along the same path (e.g., throwing a ball, etc.). For example, when a user moves the user engagement structure 104 along a path that mimics throwing a ball, the user engagement structure 104 may not cross the path until the user engagement structure 104 returns to a starting point of the path (e.g., region 202a). A repetition is counted when the user engagement structure 104 returns to a physical location that the user engagement structure 104 has already passed through during the repetition. In some examples, the repetition is only counted when the user engagement structure 104 returns to the first region 202a, regardless of whether the user engagement structure 104 has traveled through other existing regions. In some examples, the repetition is only counted when the user engagement structure 104 returns to the first region 202a or a location directly adjacent to the first region 202a, such that a buffer zone is created around the first region 202a. In this way, a repetition may still be counted even if a user slightly overshoots or undershoots the original starting location (e.g., the first region 202a).

After a repetition is counted, another path is plotted as the user moves the user engagement structure 104 for another repetition, and this subsequent path is used to determine when this next repetition should be counted (e.g., based on the subsequent path). In some examples, a new starting region is defined for each new repetition. In some examples, the starting region for a subsequent repetition is assigned to the starting region of a prior repetition (e.g., the first region 202a), regardless of where the user engagement structure 104 is located when the first repetition is counted. For example, if a first repetition is counted when the user engagement structure 104 is positioned at a location directly adjacent to the first region 202a (e.g., within the buffer zone, such as the region 202b), the starting region remains at the region 202a rather than shifting to region the 202b.

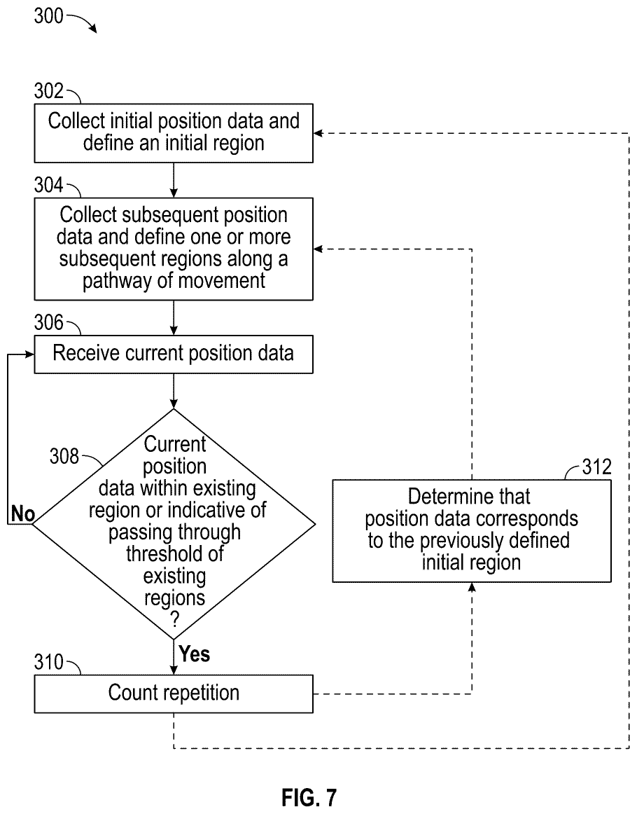

An example method 300 of counting a repetition based on the user engagement structure 104 returning to a physical space associated with an existing region is shown in FIG. 7. Using exercise device 100 as an example, the computing system 120 can receive initial position data from the position sensors 118a, 118b, 118c and an initial region (e.g., the region 202a) can be defined (process block 302). In some examples, the initial region can be defined based on coordinates of the initial position data. In some examples, the initial region can be pre-defined and it can be determined that the coordinates of the initial position data are within the initial region. After step 302, additional position data (that is, position data collected subsequent to the initial position data) can be used to plot position coordinates of the user engagement structure 104 through three-dimensional space to define one or more subsequent regions along a pathway of movement (e.g., the region 202b and/or the region 202b and one or more other regions 202 along the path 200) (process block 304). In some examples, the computing system 120 can determine regions around the position coordinates that represent and/correspond to the physical areas through which the user engagement structure 104 has traveled during the exercise (including e.g., the regions 202 that define the pathway of movement 200). As the regions of the pathway are defined, the computing system 120 can continue to collect current (and/or active) position data from the position sensors 118a, 118b, 118c of the user engagement structure 104 (process block 306).

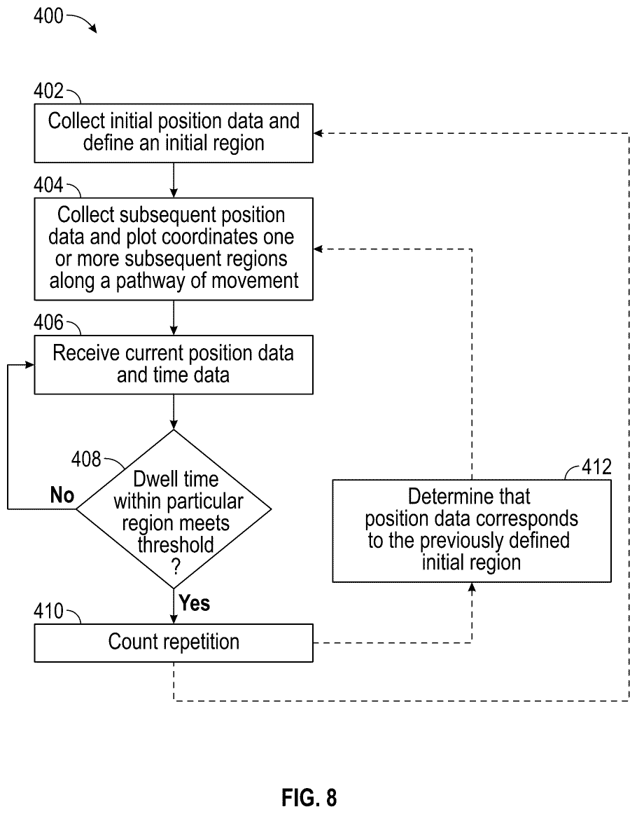

The computing system 120 can utilize the current position data to determine whether the most recent or current position coordinates are within an existing region or are indicative of passing through a threshold of existing regions (process block 308). In some examples, the active/current position coordinates can be compared to the regions that have been previously defined to determine whether they are within an existing region. In some examples, a repetition can be considered complete and is counted when the indicator 204 returns to or passes through a certain number of existing regions (e.g., two regions, three regions, etc.). In some examples, a repetition can be counted when the indicator 204 returns to or passes through a specific existing region (e.g., the original starting region, a region within a buffer zone surrounding the original starting region, etc.). In some examples, a repetition can be counted when the indicator 204 has returned to or passes through a certain percentage of the existing regions (e.g., 10% of the existing regions, 25% of the existing regions, etc.). This can be useful for exercises requiring a movement path that at least partially overlaps with itself (e.g., a FIG. 8 movement path, etc.).

If it is determined that the current position coordinates are not within an existing region or are not indicative of passing through a threshold of existing regions (NO at process block 308), the computing system 120 continues to receive and/or track current position data (process block 306). If it is determined the current position coordinates are within an existing region or are indicative of passing through a threshold of existing regions (e.g., indicating the user engagement structure 104 has returned to a location in physical space that it has already passed through) (YES at process block 308), the computing system 120 can count a repetition for the exercise (process block 310), for example, by adding one to a total repetition count. As discussed above, in some examples after the repetition is counted, a new initial region can be defined (process block 302). Alternatively, in some examples, it can be determined that current and/or active position data corresponds to the previously defined initial region (and/or a region adjacent to the initial region) (process block 312), and subsequent position data can be used to define subsequent regions along a pathway of movement starting from the previously defined initial region (process block 304).

In some examples, a repetition can be counted by the computing system 120 based on the dwell time of the indicator 204 within a particular region (e.g., within the active region 202c in FIG. 6). This can indicate that the user engagement structure 104 has reached an end point along the path and/or returned to a starting point of the next repetition. For example, the computing system 120 can utilize the time data associated with the position data from the sensors 118a, 118b, 118c to evaluate the length of time in which the indicator 204 (representative of the user engagement structure 104) is within a particular active region 202. In some examples, a repetition may be counted when the indicator 204 is within the particular active region 202 for a time that is greater than or equal to a threshold dwell time (e.g., 2 seconds, 5 seconds, etc.). In some examples, the threshold dwell time can depend on the length of time that the user has taken to move the user engagement structure 104 along the entire path (e.g., a lower threshold for shorter duration movements or a higher threshold for longer duration movements, etc.).

An example method 400 of counting a repetition based on a dwell time of the user engagement structure 104 within a particular region is shown in FIG. 8. Using exercise device 100 as an example, the computing system 120 can receive initial position from the position sensors 118a, 118b, 118c and an initial region (e.g., the region 202a) can be defined (process block 402). In some examples, the initial region can be defined based on coordinates of the initial position data. In some examples, the initial region can be pre-defined and it can be determined that the coordinates of the initial position data are within the initial region. After step 402, additional position data (that is, position data collected subsequent to the initial position data) can be used to plot position coordinates of the user engagement structure 104 through three-dimensional space (process block 404). In some examples, the computing system 120 can determine regions around the position coordinates that represent and/or correspond to the physical areas through which the user engagement structure 104 has traveled during the exercise (including e.g., the regions 202 that define the pathway of movement 200). As the regions of the pathway are defined, the computing system 120 can continue to collect current (and/or active) position data from the position sensors 118a, 118b, 118c of the user engagement structure 104 and time data associated with the current position data (e.g., timestamp data) (process block 406).

The computing system 120 can utilize the current position data and the time data to determine whether a dwell time within a particular active region is greater than or equal to a threshold (process block 408). For example, the computing system 120 can measure a dwell time of the user engagement structure 104 within the active region (that is, a time that has elapsed since the current position data entered the active region), and the computing system 120 can compare the dwell time to a pre-determined threshold (e.g., 2 seconds, 5 seconds, etc.). In some examples, the particular active region can be the initial region (e.g., the region 202a) or a region adjacent to the initial region (e.g., the region 202b). In some examples, the particular active region can be an end region or a region adjacent to the end region along the pathway 200.

If it is determined that the dwell time does not meet the threshold (NO at process block 408), the computing system 120 continues to receive and/or track current position data and time data (process block 406). If it is determined that the dwell time within the active region meets the threshold (YES at process block 408), the computing system 120 can count a repetition for the exercise (process block 410), for example, by adding one to a total repetition count. As discussed above, in some examples after the repetition is counted, a new initial region can be defined (process block 402). Alternatively, in some examples, it can be determined that current and/or active position data corresponds to the previously defined initial region (and/or a region adjacent to the initial region) (process block 412), and subsequent position data can be used to define subsequent regions along a pathway of movement starting from the previously defined initial region (process block 404).

In some examples, the computing system 120 can use a combination of both dwell time and return to an existing region to determine whether to count a repetition. For example, the computing system 120 can count a repetition when the dwell time within existing region(s) or a specified existing region (e.g., an initial region or a region adjacent to an initial region) is above a certain threshold time.

The size of the regions 202 can be customizable based on the granularity needed to determine whether a repetition has been completed. For example, when the path of the exercise is more complex (e.g., an exercise requiring a reversal of direction), the size of the regions 202 can be smaller. For exercises that define a simpler movement path (e.g., a bicep curl), the size of the regions 202 can be larger. In some examples, the original starting location of the path (e.g., the first region 202a) can have a larger size than the other regions 202, such that a buffer zone is created that allows a repetition to be counted when a user slightly overshoots or undershoots the original starting location (e.g., the first region 202a).

As shown in FIG. 6, each of the regions 202 comprises a spherical shape having a radius R. In the illustrated example, each region 202 has the same size, although, as described above, the size of one or more of the regions 202 can be adjusted based on the complexity of the movement and/or to allow greater variance in whether a movement counts as a repetition,. Based on the position data, an initial region 202a is defined with the position coordinates as the center of the sphere. For newly created regions (e.g., when the indicator 204 exits the active region), the current position coordinates define the point at which the active region and the new region are tangent. In particular, a new region is defined that is tangent to the previously active region and this point of tangency is located at the current position coordinates (e.g., the location of the indicator 204). Because a spherical shape is used for the regions 202, the two spheres are tangent at one point. In some examples, other three-dimensional shapes can be used to define the regions 202 (e.g., cubes, etc.).

In some examples, a new region 202 is defined each instance in which the indicator 204 exits the previously active region. In these examples, the newly defined region 202 is compared to the existing regions (e.g., the regions 202a,202b, or other existing regions 202). If the newly defined region overlaps with at least one of the existing regions, a repetition is counted as the exercise is considered to be complete. In some examples, a repetition is counted if multiple of the newly defined regions overlap with existing regions (e.g., a certain total number of overlapping regions, a certain total percentage of overlapping regions, a certain number of overlapping regions in a row, etc.). Regions can be considered to overlap when any portion of a new region overlaps with an existing region or based on a percentage of overlap satisfying a threshold (e.g., greater than 50% overlap between new and existing regions is considered sufficient overlap to count a repetition).

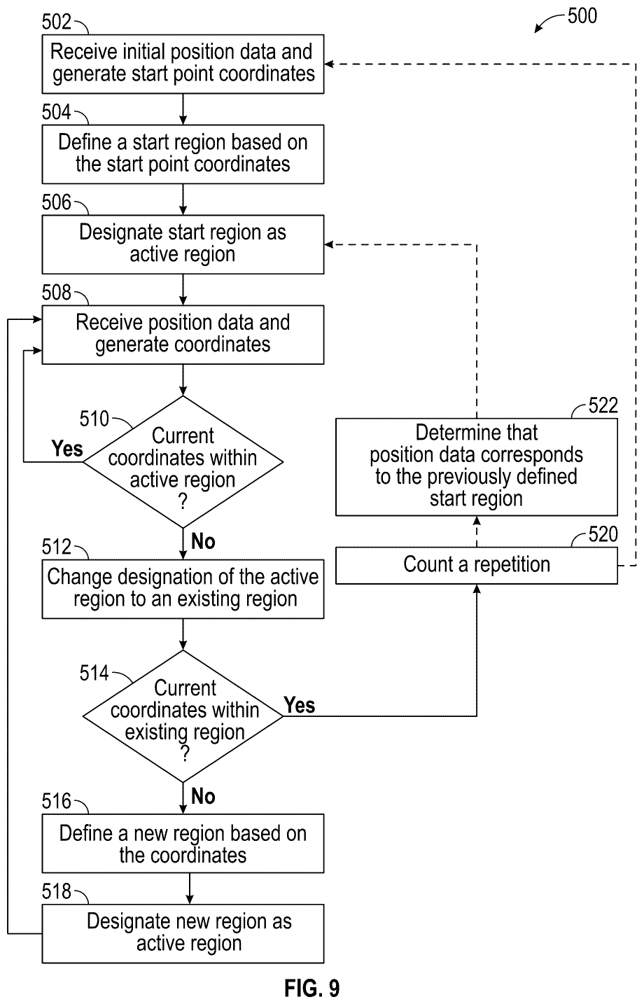

An example method 500 of counting repetitions is shown in FIG. 9. A system (such as, for example, the computing system 120) can receive position data from sensors 118a, 118b, 118c and generate position coordinates (process block 502) representative of an initial or start location of a user engagement structure of an exercise device at a first time (e.g., time T0). The computing system can define a start region based on the start point coordinates (process block 504) that includes or surrounds the coordinates. For example, the start region can have a spherical shape with the start point coordinates as the origin of the sphere. Because the coordinates are within the start region, the system can designate the start region as an active region (process block 506). The system can receive position data for a subsequent position of the user engagement structure (e.g., at time T1,) and generate subsequent position coordinates (process block 508), based on position data that is output from the sensors 118a, 118b, 118c for the subsequent position. The computing system can receive the position data from the sensors 118a, 118b, 118c in real time or near real time, such that the coordinates are generated upon receiving the position data or shortly thereafter.

The computing system can compare the subsequent position coordinates to the active region to determine whether the coordinates are within the active region (process block 510). When the subsequent position coordinates are within the active region (YES at process block 510), no new region is created, and the method 500 returns to process block 508 and can receive subsequent position data (e.g., at time T2).

If the computing system determines that the subsequent position coordinates are outside of the active region (NO at process block 510), the system changes the designation of the active region to an existing region (process block 512), to deactivate the region. The computing system can further evaluate and/or determine whether the subsequent position coordinates are within any of previously defined existing regions (process block 514). This determination can occur before or after the previously active region is deactivated as it is already known that the coordinates are not within that region.

If the system determines that the subsequent position coordinates are not within any of the existing regions (NO at process block 514), the computing system defines a new region based on the subsequent position coordinates (process block 516). In some examples, the new region is adjacent to the previously active region and the subsequent position coordinates are within the new region. The new region represents where the user engagement structure is currently located, and the computing system designates the new region as the active region (process block 518). The method 500 can then return to process block 508 and can repeat for each position that is subsequently received from the sensors over time (e.g., T3, T4, T5, etc.). In this way, multiple existing regions can be created and/or defined as the user engagement structure is moved through space along a path during an exercise (e.g., the regions 202 along the path 200 in FIG. 6). At any given time, there is only one active region, which corresponds to the region in which the user engagement structure is located (e.g., the region 202c in FIG. 6).

If the system determines that the subsequent position coordinates are within any of the previously defined existing regions (YES at process block 514), the computing system can determine that the user engagement structure 104 has re-entered an existing region along the pathway of movement and can count a repetition for the exercise (process block 520), for example, by adding one to a total repetition count. As discussed above, in some examples after the repetition is counted, a new initial region can be defined (process block 502). Alternatively, in some examples, it can be determined that current and/or active position data corresponds to the previously defined initial region (and/or a region adjacent to the initial region) (process block 522), and the previously defined start region can be designated as the active region (process block 506).

In some examples, repetitions can be counted using a vector approach, alone or in combination with the region-based approach described above. For example, a repetition can be counted when at least one of the repetition counting approaches described herein indicates that a repetition should be counted (e.g., based on one of entering an existing region, passing through a threshold of existing regions, meeting a threshold dwell time in a particular region, etc.). In some examples, the approach to repetition counting can vary based on the exercise performed by the user (e.g., the repetition counting approach is predetermined based on the particular exercise).

To determine whether a user has completed a repetition of a given exercise using the vector approach, position data from the sensors 118a, 118b, 118c can be used to calculate one or more vectors associated with the user engagement structure 104 during movement along the path (e.g., for each sample of position data received by the computing system 120). As described above, the computing system 120 can be configured to calculate vectors (e.g., position, velocity, acceleration vectors) based on the position data detected by the position sensors (e.g., the position sensors 118a, 118b, 118c). In some examples, the vectors can be further based on time data (or other data) associated with the position data. In some examples, a position vector, a velocity vector, and an acceleration vector can be calculated for each sample of position data received by the computing system 120 from the position sensors (e.g., the position sensors 118a, 118b, 118c). In some examples, the computer system 120 can normalize one or more of the calculated vectors prior to evaluating the vectors.

Initially, a start point along an exercise movement path (e.g., the path 200) for a repetition is established. The start point can comprise a data point (or coordinates) representative of the position of the user engagement structure 104 in the three-dimensional space at a particular time. In some examples, a first region (e.g., the first region 202a) can be defined around the start point or initial position of the path (e.g., the path 200). After the start point is determined, the computing system 120 evaluates whether the user engagement structure 104 is accelerating away from the start point and/or the first region based on the acceleration vector (e.g., a non-zero acceleration vector, etc.).

If the acceleration is determined to be increasing, the vectors for each subsequent sample of position data along an exercise movement path (e.g., the path 200) are evaluated to determine whether the user engagement structure 104 is returning towards the start point and/or the first region. In some examples, the determination of the return of the user engagement structure 104 towards the start point and/or the first region 202a is indicative of a repetition and the computing system 120 counts a repetition.

Specifically, for each data sample taken by the position sensors 118a, 118b, 118c and received by the computing system 120, the computing system 120 evaluates a moving average of the normalized position and velocity vectors. For example, for each data sample, the computing system 120 can take a dot product of the position vector and the velocity vector. In doing so, the computing system 120 can maintain a sample count to ensure the sample count matches for both averaged vectors. Then, for each sample, the computing system 120 compares the dot product of the current sample to a dot product of the previous sample. When the comparison indicates a reversal of direction of the user engagement structure 104 (e.g., a zero crossing, a sign change between the current dot product and the previous dot product, etc.), a repetition is counted.

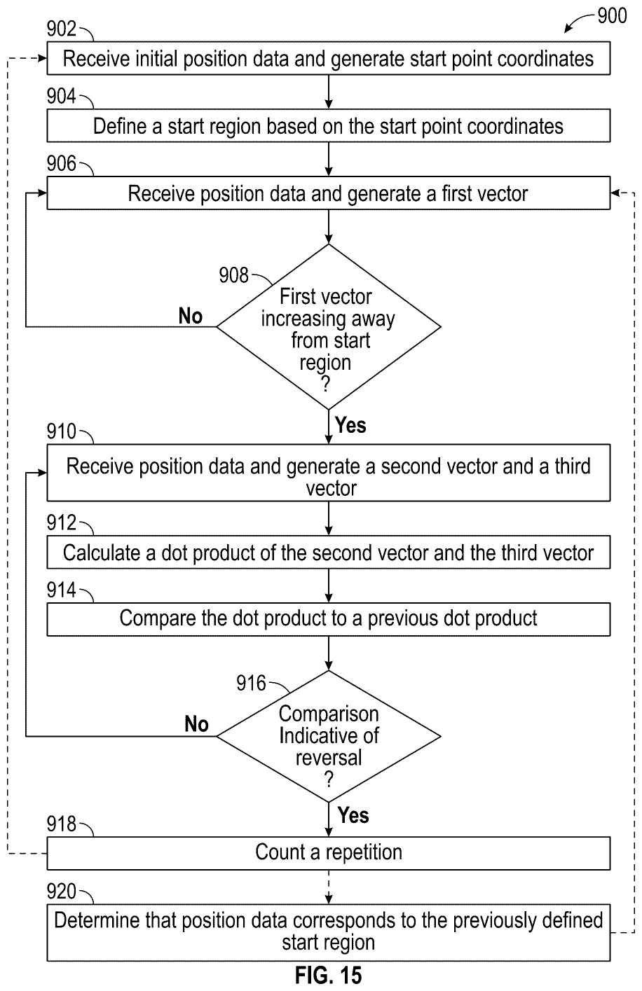

An example method 900 of counting repetitions using the vector approach is shown in FIG. 15. A computing system (e.g., the computing system 120) can receive position data from sensors 118a, 118b, 118c and generate position coordinates (process block 902) representative of an initial or start location of a user engagement structure of an exercise device. The system can define a start region based on the start point coordinates (process block 904) that includes and/or surrounds the coordinates. For example, the region can have a spherical shape with the start point coordinates as the origin of the sphere. In some examples, the start point coordinates can serve as the start region, such that the region is a single point in three-dimensional space, rather than a region.

After the start region is defined, the system can receive position data and generate a first vector (e.g., an acceleration vector) (process block 906) based on the received position data. The system can then determine whether the first vector is increasing (e.g., accelerating) away from the start region (process block 908). If it is determined that the vector is not increasing (NO at process block 908), the method repeats process blocks 906 and 908 until the system determines an increase.

After the computing system determines there is an increase away from the start region (YES at process block 908), the system continues to receive position data to evaluate whether a repetition should be counted. Specifically, for each subsequently received data sample, the computing system can generate a second vector (e.g., a velocity vector) and a third vector (e.g., a position vector) (process block 910), and can calculate a dot product of the second and third vectors (process block 912). Thereafter, the calculated dot product is compared to a previous dot product (process block 914). In particular, the previous dot product is a dot product calculated based on the position data received immediately prior to the current position data being evaluated. For example, for the first dot product comparison, the “previous” dot product is a dot product calculated based on the position data that indicated that acceleration is increasing.

In some examples, the first, second, and third vectors are determined in one process step for a sample of received position data. In some examples, the first, second, and third vectors each represent a different vector related to one sample of received position data. For example, the first vector is an acceleration vector of the received position data, the second vector is a position vector of the received position data, and the third vector is a velocity vector of the received position data. It should be appreciated that other vectors based on the received position data can be used for each of the first, second, and third vectors.

Next, the system determines whether the comparison indicates a reversal or a return of the user engagement structure 104 towards the start region (process block 916). In some examples, a reversal is indicated when the current dot product has a different sign than the previous dot product (e.g., a sign change). If it is determined that the comparison does not indicate a reversal (NO at process block 916), the method returns to process block 910. If it is determined that the comparison indicates a reversal (YES at process block 916), a repetition is counted (process block 918), for example, by adding one to a total repetition count. As discussed above, in some examples after the repetition is counted, a new initial region can be defined (process block 902). Alternatively, in some examples, it can be determined that current and/or active position data corresponds to the previously defined initial region (and/or a region adjacent to the initial region) (process block 920), and subsequent position data can be used to generate a first vector and determine whether the first vector is moving away from the previously defined start region (process blocks 906 and 908).

### Resistance

The resistance applied by the resistance mechanism 114 can be controlled by the computing system 120 such that resistance experienced by the user is constant and balanced throughout the exercise (e.g., constant along path 200, etc.). For example, regardless of the position and/or length of the shaft assembly 102, the computing system 120 can control the amount of braking force applied by each brake (e.g., brakes 114a, 114b, 114c) based on data from the resistance sensors 116a, 116b, 116c.

In some examples, the resistance experienced by the user is a function of the input from the user to the exercise device 100 (e.g., an amount of force applied by the user to the shaft assembly 102, etc.) as well as the resistive or braking force that is being applied by the brakes 114a, 114b, 114c. For example, a braking force can be applied to the xy-plane by the brakes 114a and 114b and a braking force can be applied in the z direction, in part, by the brake 114c. The resistance experienced by the user can be calculated by the computing system 120 based on the measurements taken by the resistance sensors 116a, 116b, 116c (e.g., the four pressure sensors 116a, 116b and the load cell 116c). The resistance values may be factored to the distance the telescoping shaft 106 is extended from the base 108 (e.g., a distance the telescoping shaft 106 is extended from the base 108 along the axis A3). For example, in some instances, the shaft assembly 102 may be easier to move when the telescoping shaft 106 is in an extended position, such that a higher resistance or braking force is applied by the resistance mechanism 114. In this way, even though the brakes 114a, 114b, 114c may be located at a proximal end of the shaft assembly 102, the resistance experienced by the user at the distal end of the shaft assembly 102 (e.g., at the user engagement structure 104) may be determined.

The braking force applied by the resistance mechanism 114 can be dynamically changed based on the positioning of the shaft assembly 102 and the actual braking loads as detected by the resistance sensors 116a, 116b, 116c. In this way, the resistance sensors 116a, 116b, 116c enable the resistance mechanism 114 to react to the actual braking loads (e.g., in real time or near real time) to maintain an accurate and balanced resistance on the shaft assembly 102. In particular, the computing system 120 can utilize a controller configured to adjust the resistance applied by the brakes 114a, 114b, 114c to maintain equal resistance (e.g., using motor control theory). In some examples, the computing system 120 can utilize a controller, such as a proportional integral derivative (PID) loop or other controllers, to automatically and dynamically adjust the resistance applied by one or more of the brakes 114a, 114b, 114c to balance the resistance experienced by the user when moving the shaft assembly 102.

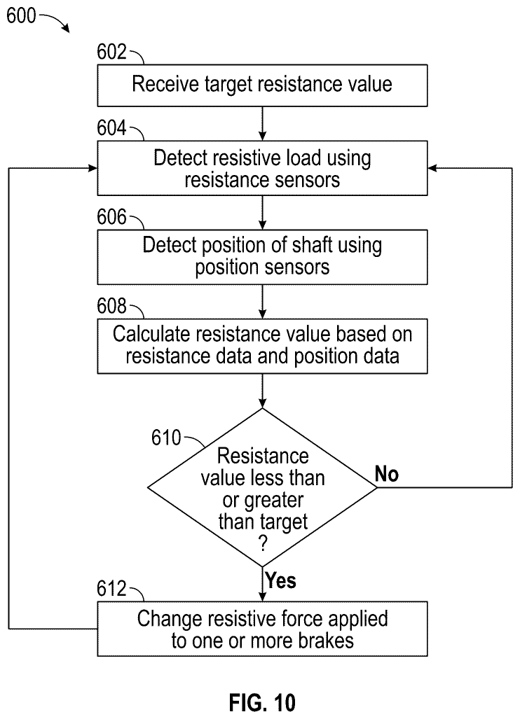

FIG. 10 illustrates an example method 600 of dynamically adjusting resistance of an exercise device. The following description uses exercise device 100 as an example, although the method can be used with other exercise devices. Initially, the computing system 120 receives a target resistance value (process block 602). The target resistance value can be selected by an operator (e.g., a user or a health practitioner), for example, when the operator selects an exercise and/or prior to using the exercise device 100. In some examples, the target resistance can be predetermined based on the exercise, a rehabilitation and/or training program, or the like. For example, in a rehabilitation program, the resistance value can be low or zero to provide a weightless feel to the user as the user moves the shaft assembly 102 during an exercise.

The resistance sensors 116a, 116b, 116c can detect a resistive load experienced at each of the resistance sensors 116a, 116b, 116c and transmit the resistance data to the computing system 120 (process block 604). The position sensors 118a, 118b, 118c can detect the position of the user engagement structure 104 of the shaft assembly 102 and transmit the position data to the computing system 120 (process block 606). Based on the resistance and position data, the computing system 120 can calculate a resistive load experienced by the user at the user engagement structure 104 (process block 608).

The computing system 120 can adjust the resistive loads applied by the brakes 114a, 114b, 114c to keep the resistance (e.g., as calculated by the computing system 120 based on the data from the resistance sensors 116a, 116b, 116c and/or the position sensors 118a, 118b, 118c) at or within a range of the target resistance. For example, the computing system can determine whether the calculated resistance value is less than or greater than the target resistance value (process block 610). If the calculated resistance value does not match the target resistance value (YES at process block 610), the computing system can change the resistance force applied by one or more of the brakes 114a, 114b, 114c (process block 612). In some examples, the resistance force can be changed when difference between the calculated and target values is above a threshold. If the calculated resistance value does match the target resistance value (NO at process block 610), then the method returns to process block 604 so that the computing system continues to detect the resistive load over time and/or duration of the exercise.

The method 600 can be performed as an iterative loop by the computing system 120, for example, to adjust the resistive force applied by the brakes 114a, 114b, 114c as additional position and resistance data are received from the resistance sensors 116a, 116b, 116c and/or the position sensor118a, 118b, 118c. For example, the computing system 120 can repeat process blocks 604-612 throughout use of the exercise device 100 and/or selectively during particular uses of the exercise device 100 (e.g., when the shaft assembly 102 is in motion).

In some examples, the computing system 120 can selectively determine when to dynamically adjust the resistance applied by the resistance mechanism 114 (e.g., only when the shaft assembly 102 is in motion). For example, if the computing system 120 determines that the shaft assembly 102 is stationary, the computing system 120 can keep the resistance the same. However, when the computing system 120 detects that the shaft assembly 102 is in motion (e.g., based on position sensors 118a, 118b, 118c), the computing system 120 can dynamically adjust the resistance, for example, using a PID control loop as described above.

The exercise device 100 can be configured with an automatic braking feature, in some examples. For instance, the computing system 120 can detect when the user engagement structure 104 makes a rapid departure from the projected path (e.g., based on position data and/or data based on or associated with the position data, such as position coordinates, time, velocity, acceleration, etc.) and cause the resistance mechanism 114 to apply a sufficiently high braking force (e.g., a higher resistance than was previously applied) to slow down or cease motion of the shaft assembly 102. In some instances, the braking force can be applied when the computing system 120 detects that the user is falling and/or to prevent injury of the user. For example, if the velocity of the shaft assembly 102 is above a certain threshold and/or the computing system 120 detects a departure from the projected path for a given exercise, the resistance mechanism 114 can increase the braking force applied to the shaft assembly 102.

### Performance Metrics

Based on data obtained by sensors of the exercise device 100 (e.g., position data, force data, etc.), performance metrics can be determined by the computing system 120. For example, an accuracy metric can be determined based on the accuracy with which a user completes a given exercise. In some examples, the accuracy can be based on the physical closeness to which the user follows a particular path, the velocities obtained by the user while performing an exercise, and/or the force a user imparts while performing an exercise.

The exercise device 100 can determine an accuracy of a given repetition of an exercise as compared to a reference path for that exercise. In some examples, a reference path for an exercise can be predetermined, for example, by a professional or physical therapist. In this way, the predetermined path for the exercise can represent an ideal path that uses proper form to complete the exercise.

In some examples, a reference path can be defined by the user, for example, under the supervision of a professional or physical therapist, such that the reference path is specific to the user (e.g., size and/or mobility of the specific user, etc.). In this way, the user defined reference path can be used as a baseline for the user that can be to compare subsequent repetitions, including, for example, while the user performs a training and/or rehabilitation program without supervision by a professional or physical therapist. In some examples, a reference path can be based on the first path of an exercise as executed by the user.

In some examples, the reference path for a given exercise can be stored by the computing system 120 and selected by a user prior to performing the exercise (e.g., as part of an exercise and/or rehabilitation regime). The computing system 120 can compare subsequent repetitions of the exercise to the reference path for the exercise. For example, data associated with each repetition can be compared, including the position coordinates for the path, force measurements, time values, velocity, acceleration, and/or other data.

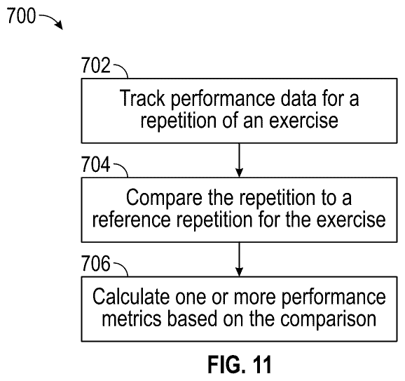

An example method 700 of evaluating performance metrics is shown in FIG. 11. The computing system 120 can track performance data for a repetition of an exercise (process block 702). The performance data can be position data, time data, resistance data, and/or data based on or associated with such data (e.g., velocity, acceleration, force, power, etc.). The performance data for the repetition can be compared to performance data associated with a reference repetition (e.g., a reference path) for the exercise (process block 704) and a performance metric (e.g., any metric described herein, such as an accuracy metric, a fatigability metric, etc.) can be calculated based on this comparison (process block 706).

To compare the accuracy of exercise movements to the reference path, the computing system 120 can project the path associated with the completed exercise movement onto the reference path. Accuracy values can be created by comparing the position coordinates, force values, time, velocity, and/or acceleration values of the reference path against those of the completed path. For example, the computing system 120 can determine an accuracy metric for the repetition based physical location based on a comparison of the path/position (x, y, z) coordinates at each recorded time.

In some examples, to compare the reference path against a completed path, the computing system 120 is configured to compare the three-dimensional paths by taking multiple two-dimensional views of the paths and comparing pixels. For example, a comparison of the paths can be performed by assessing the different values (e.g., position, force, time, etc.) from a view taken in the x-direction, y-direction, z-direction, or some other view.

The computing system 120 can also be configured to calculate a strength metric based on the power generated by the user during performance of an exercise. In some examples, the computing system 120 can determine a strength metric for the repetition based on the resistance applied by the resistance mechanism 114 and the distance the shaft assembly 102 has moved over time (e.g., indicative of a power value). For example, the computing system 120 can use data obtained by the resistance sensors 116a, 116b, 116c and/or the position sensor118a, 118b, 118c as inputs into the power equation to determine a strength metric and/or other performance metrics. In some examples, the strength metric can be indicative of a maximum power value, a minimum power value, an average power value, etc.

In some examples, the computing system 120 can determine a fatigability metric based on the rate of movement (e.g., velocity) over time for a given exercise. For example, using the position data and/or the resistance data over time, the computing system 120 can calculate a rate of movement associated with a repetition of an exercise (e.g., an average velocity, a maximum velocity, etc.) and detect when the rate of movement decreases, for example, after a certain number of repetitions. The fatigability metric can be evaluated over multiple training and/or rehabilitation sessions to monitor an endurance of the user.

Another useful metric in evaluating shoulder strength is the ability of a user to reverse directions. This reversal metric can be based on a change of momentum, direction, and/or acceleration as detected by the sensors of the exercise device 100 (e.g., using position data, time data, and/or resistance data as measured by the resistance sensors 116a, 116b, 116c and/or the position sensor118a, 118b, 118c). Traditional shoulder rehabilitation exercise equipment, such as bands, can be biased in a particular direction, such that the bands encourage the user to move in the opposite direction (e.g., return to the starting position). Because the resistance applied by the exercise device 100 can be neutral and balanced in multiple directions as described above, the exercise device 100 can enable the user to reverse directions without bias, allowing such movement to be measured and evaluated by the exercise device 100.

In some examples, data obtained from the exercise device 100 can be used to determine a range of motion metric (e.g., for a shoulder, etc.). For example, a user can be guided through a series of exercises using the exercise device 100 which can be used by the computing system 120 to calculate a position of the shoulder of the user and/or an arm length of the user. In some examples, the arm length or other size parameters of a user can be entered into the user's profile and used by the computing system 120 to determine the shoulder positioning. Based on the determination of shoulder location, subsequent movements of the user engagement structure 104 (e.g., representative of a hand position) can be used to evaluate a range of motion (e.g., based on position data from the position sensors 118a, 118b, 118c).

The performance metrics can be stored by the computing system 120 to a profile associated with the user. In some examples, the performance metrics can be used to determine whether the user has made improvements over time, for example, to determine whether the user is completing various exercises indicative of the user getting stronger and/or faster.

### Example Computing System

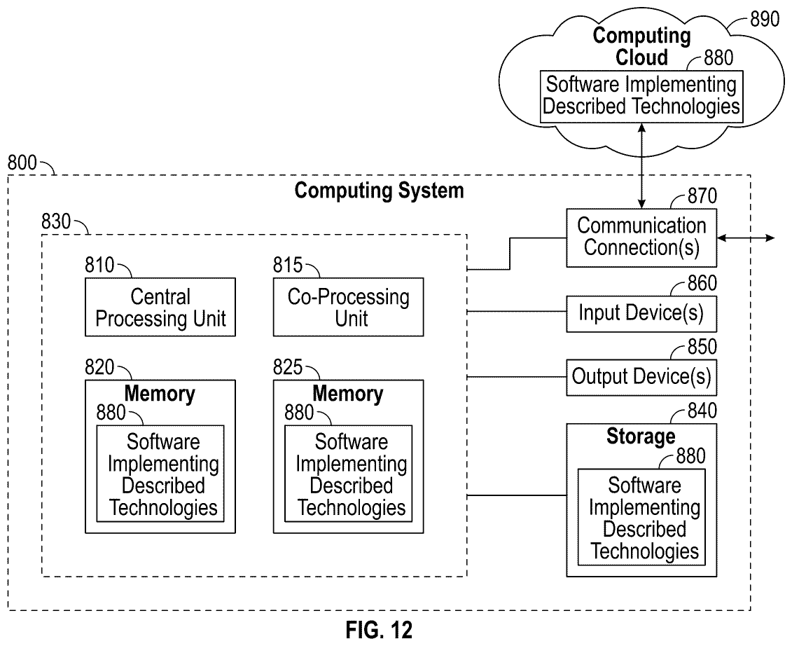

FIG. 12 depicts a generalized example of a suitable computing system 800 in which the described innovations may be implemented. The computing system 800 is not intended to suggest any limitation as to scope of use or functionality, as the innovations may be implemented in diverse general-purpose or special-purpose computing systems. For example, the computing system 800 can be used to implement hardware and software.

With reference to FIG. 8, the computing system 800 includes one or more processing units 810, 815, non-volatile memory 820, and memory 825. In FIG. 8, this basic configuration 830 is included within a dashed line. The processing units 810, 815 execute computer-executable instructions, including instructions for generating shape models, locating fiducials in images, and/or aligning an output device with an article as disclosed herein. A processing unit can be a general-purpose central processing unit (“CPU”), processor in an application-specific integrated circuit (“ASIC”), or any other type of processor. In a multi-processing system, multiple processing units execute computer-executable instructions to increase processing power. For example, FIG. 8 shows a central processing unit 810 as well as a graphics processing unit (“GPU”) or co-processing unit 815. The tangible memory 825 may be volatile memory (e.g., registers, cache, RAM), non-volatile memory (e.g., ROM, EEPROM, flash memory, etc.), or some combination of the two, accessible by the processing unit(s). The memory 825 stores software 880 implementing one or more innovations described herein, in the form of computer-executable instructions suitable for execution by the processing unit(s).

A computing system may have additional features. For example, the computing system 800 includes storage 840, one or more input devices 850, one or more output devices 860, and one or more communication connections 870. An interconnection mechanism (not shown) such as a bus, controller, or network interconnects the components of the computing system 800. Typically, operating system software (not shown) provides an operating environment for other software executing in the computing system 800, and coordinates activities of the components of the computing system 800.

The tangible storage 840 may be removable or non-removable, and includes magnetic disks, magnetic tapes or cassettes, CD-ROMs, DVDs, or any other medium which can be used to store information and which can be accessed within the computing system 800. The storage 840 stores instructions for the software 880 implementing one or more innovations described herein.

The input device(s) 850 may be a touch input device such as a keyboard, mouse, pen, or trackball, a voice input device, a scanning device, microphone, button, pedal, or another device that provides input to the computing system 800. For video encoding, the input device(s) 850 may be a camera with an image sensor, video card, TV tuner card, or similar device that accepts video input in analog or digital form, or a CD-ROM, CD-RW, DVD, or Blu-Ray that reads video samples into the computing system 800. The output device(s) 860 may be a display, printer, speaker, CD-writer, or another device that provides output from the computing system 800.

The communication connection(s) 870 enable communication over a communication medium (e.g., a connecting network) to another computing entity. The communication medium conveys information such as computer-executable instructions, compressed graphics information, video, or other data in a modulated data signal. The communication connection(s) 1170 are not limited to wired connections (e.g., megabit or gigabit Ethernet, Infiniband, Fibre Channel over electrical or fiber optic connections) but also include wireless technologies (e.g., RF connections via Bluetooth, WiFi (IEEE 802.11a/b/n), WiMax, cellular, satellite, laser, infrared) and other suitable communication connections for providing a network connection for the disclosed agents, bridges, and agent data consumers. In a virtual host environment, the communication(s) connections can be a virtualized network connection provided by the virtual host.

Some embodiments of the disclosed methods can be performed using computer-executable instructions implementing all or a portion of the disclosed technology in a computing cloud 890. For example, disclosed computer-readable instructions can be executed by processors located in the computing environment 830, or the disclosed computer-readable instructions can be executed on servers located in the computing cloud 890.

Computer-readable media are any available media that can be accessed within a computing system 800. By way of example, and not limitation, with the computing system 800, computer-readable media include memory 820 and/or storage 840. As should be readily understood, the term computer-readable storage media includes the media for data storage such as memory 820 and storage 840, but does not include transmission media such as modulated data signals or other transitory signals.

The innovations can be described in the general context of computer-executable instructions, such as those included in program modules, being executed in a computing system on a target real or virtual processor. Generally, program modules include routines, programs, libraries, objects, classes, components, data structures, etc. that perform particular tasks or implement particular data types. The functionality of the program modules may be combined or split between program modules as desired in various embodiments. Computer-executable instructions for program modules may be executed within a local or distributed computing system.

In some embodiments, the computing system 120 can be configured with components and/or functionality of computing system 800.

In view of the many possible embodiments to which the principles of the disclosed technology may be applied, it should be recognized that the illustrated embodiments are only examples of the technology and should not be taken as limiting the scope of the technology. Rather, the scope of the technology is defined by the following claims and their equivalents.

1. A computerized method for counting a repetition of an exercise utilizing an exercise device comprising a user engagement structure and a position sensor configured to detect a position of the user engagement structure, the method comprising:
for each of a plurality of positions of the user engagement structure:
receiving position data from the position sensor during the exercise;
generating one or more vectors based on the position data;
comparing the one or more vectors to one or more prior vectors; and
counting a repetition of the exercise when the comparison of the one or more vectors to the one or more prior vectors indicates the user engagement structure is returning towards one or more of a starting point or a starting region of the exercise.

2. The method of claim 1, wherein the comparing the one or more vectors to the one or more prior vectors comprises:
calculating a dot product based on the one or more vectors;
calculating a dot product based on the one or more prior vectors; and
comparing a sign of the dot product based on the one or more vectors and a sign of the dot product based on the one or more prior vectors.

3. The method of claim 2, wherein the counting the repetition of the exercise comprises:
counting the repetition of the exercise when the sign of the dot product based on the one or more vectors is different than the sign of the dot product based on the one or more prior vectors.

4. The method of claim 1, wherein the one or more vectors comprise an acceleration vector, a position vector, and a velocity vector.

5. The method of claim 1, further comprising:
receiving initial position data from the position sensor at initiation of the exercise; and
generating position coordinates representative of the starting point.

6. The method of claim 4, further comprising defining a start region based on coordinates forming a 3-dimensional shape surrounding the starting point coordinates.

7. The method of claim 6, wherein the 3-dimensional shape is one of a sphere or a cube.

8. A computerized apparatus for use with an exercise device comprising a user engagement structure and a position sensor configured to detect a position of the user engagement structure, the computerized apparatus comprising:
a communication interface configured to receive signals from the position sensor;
one or more processors; and
a memory in data communication with the one or more processors and having a plurality of computer-readable instructions stored thereon, wherein the plurality of computer-readable instructions are configured to, when executed by the one or more processors, cause the computerized apparatus to:
receive first position data from the position sensor and generate an acceleration vector based on the first position data;
determine whether the acceleration vector is increasing in a direction away from the one or more of a starting point or a starting region of an exercise;
based at least on a determination that the acceleration vector is increasing in the direction away from the one or more of the starting point or the starting region of the exercise, for each of a plurality of plurality of positions of the user engagement structure:
receive second position data from the position sensor during the exercise;
generate one or more vectors based at least on the second position data;
generate a dot product based on the one or more vectors;
compare the dot product to a previously generated dot product; and
determine whether the comparison is indicative of a reversal; and
based at least on a determination that the comparison is indicative of a reversal, count a repetition of the exercise.

9. The computerized apparatus of claim 8, wherein the acceleration vector is a first acceleration vector, wherein the one or more vectors include a position vector, a velocity vector, and a second acceleration vector.

10. The computerized apparatus of claim 8, wherein the previously generated dot product is an immediately previously generated dot product relative to the dot product.

11. The computerized apparatus of claim 8, wherein the determination that the comparison is indicative of a reversal comprises a determination of a sign reversal between the dot product and the previously generated dot product.

12. The computerized apparatus of claim 8, wherein the determination that the comparison is indicative of a reversal comprises a determination of a zero crossing between the dot product and the previously generated dot product.

13. The computerized apparatus of claim 8, wherein the plurality of computer-readable instructions are further configured to, when executed by the one or more processors, cause the computerized apparatus to:
receive initial position data from the position sensor at initiation of the exercise; and
generate initial position coordinates representative of the one or more of the starting point or the starting region of the exercise.

14. The computerized apparatus of claim 13, wherein the plurality of computer-readable instructions are further configured to, when executed by the one or more processors, cause the computerized apparatus to:
after the count of the repetition of the exercise, receive third position data from the position sensor; and
determine that the third position data is indicative of a return of the user engagement structure to the one or more of the starting point or the starting region of the exercise.

15. The computerized apparatus of claim 13, wherein the plurality of computer-readable instructions are further configured to, when executed by the one or more processors, cause the computerized apparatus to:
after the count of the repetition of the exercise, receive third position data from the position sensor; and
based at least on the third position data, generate new position coordinates representative of the one or more of the starting point or the starting region of the exercise.

16. One or more computer-readable storage media having a plurality of computer-readable instructions stored thereon, the plurality of computer-readable instructions are configured to, when executed by one or more processors, cause a computerized apparatus in communication with a position sensor of an exercise apparatus to:
define one or more of a starting point or a starting region of an exercise;
determine, based at least on first position data received from a position sensor, that a user engagement structure of the exercise apparatus is accelerating away from the one or more of a starting point or a starting region of the exercise;
based at least on the determination that the user engagement structure is accelerating away from the one or more of a starting point or a starting region of the exercise, for each of a plurality of plurality of positions of the user engagement structure:
receive second position data from the position sensor during the exercise;
generate one or more vectors based at least on the second position data;
generate a dot product based on the one or more vectors;
compare the dot product to one or more immediately previously generated dot products; and
determine whether the comparison is indicative of one or more of a sign change or a zero crossing; and
based at least on a determination that the comparison is indicative of the one or more of a sign change or the zero crossing, add one to a total count of repetitions of the exercise.

17. The one or more computer-readable storage media of claim 16, wherein the one or more vectors comprise a position vector, a velocity vector, and an acceleration vector.

18. The one or more computer-readable storage media of claim 16, wherein the plurality of computer-readable instructions are further configured to, when executed by the one or more processors, cause the computerized apparatus to:
receive initial position data from the position sensor at initiation of the exercise; and
generate initial position coordinates representative of the one or more of the starting point or the starting region of the exercise.

19. The one or more computer-readable storage media of claim 18, wherein the initial position coordinates are representative of the starting region of the exercise, wherein the starting region comprises a 3-dimensional shape surrounding the initial position coordinates.

20. The one or more computer-readable storage media of claim 16, wherein the generation of the one or more vectors is further based at least on time data associated with the second position data.
